(19) United States
(12) Patent Application Publication
(10) Pub. No.: US 2011/0137022 A1
(43) Pub. Date: Jun. 9, 2011

(54) METHOD FOR THE ANALYSIS OF OLIGONUCLEOTIDES

(76) Inventors: Dennis Paul Michaud, Dracut, MA (US); Paul McCormac, Cambridge, MA (US)

(21) Appl. No.: 12/737,771

(22) PCT Filed: Aug. 7, 2009
(86) PCT No.: PCT/GB2009/001940

§ 371 Date: Feb. 14, 2011

Related U.S. Application Data
(60) Provisional application No. 61/090,254, filed on Aug. 20, 2008.

(51) Int. Cl. C07H 1/00; B01D 59/44

(52) U.S. Cl. 536/25.34; 250/282; 536/25.3

(57) ABSTRACT

A method for the analysis of oligonucleotides is provided. The method comprises: desalting a mixture of oligonucleotides under conditions such that there is substantially no separation of the mixture of oligonucleotides, and/or by on-line desalting, introducing the desalted mixture of oligonucleotides into a mass spectrometer; and quantifying one or more oligonucleotides comprised in the mixture by mass spectrometry.

The present invention concerns a method for the analysis of oligonucleotides.

Oligonucleotides are of significant current interest in the field of pharmaceuticals. Such compounds are typically synthesised by the stepwise addition of synthons to the growing oligonucleotide chain. The basic sequence for the addition of a single nucleoside is commonly deprotect—couple—oxidise—cap. On completion of assembly of the desired sequence, protecting groups employed to prevent competing reactions during the synthesis are removed, and, in the most common situations where the oligonucleotide has been synthesised using by solid phase methods, the oligonucleotide is cleaved from the solid support. The synthesis of a typical 22-mer oligonucleotide therefore involves many individual process steps. Despite the high efficiency of modem oligonucleotide synthesis chemistry, given the number of steps involved, and the large number of potentially reactive sites present, small amounts of impurities are often formed during the synthesis. Typically such impurities comprise shorter than expected sequences due to coupling and/or capping efficiencies of less than 100%, or species comprising higher than expected molecular weights which may be caused by side-reactions. It is therefore necessary to be able to identify and quantify the levels of impurity present in the oligonucleotide product.

Methods for quantifying impurities present in oligonucleotides typically comprise HPLC chromatography with ultraviolet detection for quantification, with mass spectroscopy employed for characterisation. International patent application WO2006/107775 discloses a method where oligonucleotides are partially separated by chromatography, with quantification of separated oligonucleotides being achieved by uv analysis combined with the quantification of co-eluting oligonucleotides by mass spectrometry. This method requires time-consuming chromatographic analysis and a plurality of quantification methods. It is therefore desirable to identify alternative methods amenable to more rapid and simpler analysis.

According to a first aspect of the present invention, there is provided a method comprising:

a) desalting a mixture of oligonucleotides under conditions such that there is substantially no separation of the mixture of oligonucleotides;
b) introducing the desalted mixture of oligonucleotides into a mass spectrometer; and
c) quantifying one or more oligonucleotides comprised in the mixture by mass spectrometry.

According to a second aspect of the present invention, there is provided a method comprising:

a) on-line desalting a mixture of oligonucleotides;
b) introducing the desalted mixture of oligonucleotides into a mass spectrometer; and
c) quantifying one or more oligonucleotides comprised in the mixture by mass spectrometry.

Mixtures of oligonucleotides which can be employed in the methods of the present invention are commonly the products of solid phase chemical synthesis, preferably synthesis by the phosphoramidite approach. Such mixtures comprise a target full length oligonucleotide product as the predominant component and varying amounts of oligonucleotide impurities, for example, shortmers or failure sequences, often referred to as n-x sequences, where n is the number of nucleotides in an oligonucleotide, and x is a positive integer, where x is <n; such as n-1, n-2 and n-3 sequences; adducts such as acetyl adducts, isobutyryl adducts, chloral insertions; cyanoethyl adducts arising from acrylonitrile addition, methyl adducts and related groups; sequences lacking one or more nucleobases, for example depurinated or depyrimidinated sequences; sequences comprising extra nucleoside, often by addition to an amino group of a nucleobase; monophosphodiester impurities and similar sulphur deficit impurities in phosphorothioate oligonucleotides; and additional phosphate groups on oligonucleotides, including on shortmer or failure sequences.

Oligonucleotides which can be employed include deoxyribonucleotides, ribonucleotides and chimeric compounds comprising deoxyribo- and ribonucleotides. The oligonucleotides can be single-stranded or duplexes. One or more modifications may be present, such as phosphorothioate linkages, 2′-modifications, such as 2′-O-alkyl, especially methyl; 2′-O-alkoxyalkyl, such as methoxyethyl; 2′-C-allyl; and 2′-fluoro modifications. Abasic moieties and unnatural nucleobases, such as inosine and hypoxanthine may be present. The oligonucleotides may be in the D-configuration, the L-configuration or may comprise a mixture of D and L-nucleotides. In many embodiments, the oligonucleotides comprise from up to 70, commonly 5 to 50 nucleobases per strand, such as from 16 to 25 nucleobases per strand. In many embodiments, the mixture of oligonucleotides comprises a drug product.

Synthetic oligonucleotide mixtures typically comprise non-volatile salts, commonly inorganic salts, such as sodium salts, either as extraneous salts or as counter-ions to the oligonucleotide. Such salts interfere with analysis by mass spectroscopy. The process by which such non-volatile salts are removed from the oligonucleotide is known as desalting. Desalting can be achieved by methods known in the art, including alcohol precipitations, ion-exchange methods, size-exclusion chromatography and the use of ion-pair reverse phase liquid chromatography (“RPLC”). In order to desalt an oligonucleotide, non-volatile counter ions, commonly sodium ions, are exchanged for volatile counter ions, such as ammonium ions, and especially organic ammonium ions. Such exchange is normally achieved by contacting the oligonucleotide mixture with a solution of a salt of the volatile counter ion, preferably an ammonium salt. Examples of ammonium salts which can be employed include NH₄⁺, and primary, secondary and tertiary ammonium salts, which may comprise one or more of alkyl, aryl, alkaryl or aralkyl moieties. Preferred alkyl groups which may be present are C₁₋₈ alkyl groups, especially C₁₋₄ alkyl groups, and which may be primary, secondary or tertiary alkyl groups. Preferred aryl groups which may be present include phenyl groups. Preferred alkaryl groups which may be present include C₁₋₈alkylphenyl groups. Preferred aralkyl groups which may be present include phenylC₁₋₈alkyl

groups, especially benzyl or phenylethyl groups. Ammonium salts commonly employed include salts formed with acids, especially carbonic or carboxylic acids, such as carbonate, formate, acetate, trifluoroacetate and propionate salts. Mildly acidic volatile compounds, such as halogenated C₁₋₄ alcohols, especially polyfluorinated alcohols such as hexafluoroisopropanol may be employed to form ammonium salts.

It will be recognised that on-line desalting refers to a process where the mixture of oligonucleotides is desalted and the desalted oligonucleotide mixture then transferred to a mass spectrometer without intermediate manual intervention, and preferably without intermediate processing or treatment of the desalted mixture. The on-line desalting most preferably is achieved under conditions such that there is substantially no separation of the mixture of oligonucleotides.

Size-exclusion chromatography comprises passing a solution of the mixture of oligonucleotides through a medium, typically a porous bead, which allows material of below a certain size to enter into the medium, delaying its passage through the medium, whereas larger material passes through. In the case of desalting oligonucleotides, the non-volatile salts pass into the medium and hence are separated from the oligonucleotides.

When size-exclusion chromatography is employed to desalt, the mixture of oligonucleotides is passed through a size-exclusion chromatography medium, most commonly in the form of an aqueous solution. Flow rates and column dimensions are typically selected such that the mixture of oligonucleotides elutes in less than 10 minutes, most preferably less than 5 minutes. The elution of the oligonucleotide can be detected qualitatively if desired by in-line uv detection or preferably the eluent is passed directly to a mass spectrometer for quantitative analysis. Salts retained on the chromatography medium can be removed by washing, preferably to waste, after elution of the oligonucleotide mixture.

Ion-pair reverse phase liquid chromatography (RPLC) is a well known technique for the analysis of oligonucleotides wherein the conditions are normally selected to achieve chromatographic separation of the components of a mixture of oligonucleotides based on differential hydrophobicity. In the method of the present invention, conventional RPLC media are employed, typically alkylated silica media such as RP C-18, but the elution conditions are selected such that the oligonucleotide mixture elutes as a single peak. Conditions are selected such that on introduction of a solution of oligonucleotide into the RPLC medium, the relatively more hydrophobic oligonucleotides are retained by the RPLC medium, whereas the hydrophilic, unwanted salts pass more quickly through the column. In many embodiments, solvent conditions are varied to facilitate rapid passage of the unwanted salts with retention of the oligonucleotides, followed by elution of the oligonucleotides. A solvent gradient may be employed, but in many embodiments, it is preferred to employ a step change from the loading solvent, commonly a hydrophilic solvent, to the elution solvent, commonly a hydrophobic solvent.

When RPLC is used to desalt, the oligonucleotide mixture is preferably loaded onto the RPLC medium using an aqueous or preferably a water miscible organic solution which is predominantly aqueous. Preferably, the loading solution comprises at least 60% v/v water, commonly at least 75% v/v water, especially at least 85% v/v water and most preferably at least 90% water. Most preferably, the loading solution comprises the salt of the volatile counter-ion. After the non-volatile salts have eluted, the mixture of oligonucleotides is eluted by passing an elution solvent through the RPLC medium. Preferably, the mixture of oligonucleotides is eluted with a mixture of water and a water miscible organic solution which is predominantly organic. Preferably the elution solution comprises at least 55% v/v organic solvent, commonly at least 65% v/v organic solvent, especially at least 75% v/v organic solvent. The elution solution additionally comprises the salt of the volatile counter-ion. Water-miscible organic solvents which may be present include those commonly employed in HPLC, such as C₁₋₃ alcohols, such as methanol or ethanol and C₁₋₃nitriles. Acetonitrile is the preferred organic solvent employed in both loading and elution solutions. Most preferably, both loading and elution solutions comprise a salt of the volatile counter-ion, commonly an ammonium salt as described above, and preferably a salt comprising a trialkylamine and an acid. Preferred trialkylamines include tripropylamine, tributylamine, dimethylbutyla mine and diethylbutylamine. Preferred acids comprise carbonic acid, formic acid, acetic acid, trifluoroacetic acid and propionic acid. A buffer comprising dimethylbutylamine and acetic acid is most preferred. In certain embodiments, the mobile phase is buffered to about neutral pH, such as from 6 to 8, preferably from 6.5 to 7.5. Conditions are advantageously selected such that the mixture of oligonucleotides typically elutes in less than 10 minutes, most preferably less than 5 minutes. The eluted mixture of oligonucleotides may be passed directly to the mass spectrometer without intervening detection methods, but in many embodiments, it is preferred that the elution of the oligonucleotide is detected qualitatively, most preferably by in-line uv detection prior to quantitative mass spectral analysis.

The desalting of the mixture of oligonucleotides is carried out under conditions such that substantially no separation of the mixture of oligonucleotides occurs. Preferably at least 95%, preferably at least 96%, more preferably at least 97%, especially at least 98% and most preferably at least 99% of the oligonucleotides coelute.

In many embodiments, the desalting is carried out such that non-volatile salts, especially sodium salts, are reduced to concentrations where they do not interfere with analysis by mass spectrometry. It is preferred that the mixture of oligonucleotides is desalted to the extent that non-volatile salt, especially sodium, adducts comprise less than 20% w/w of the mixture of oligonucleotides, more preferably less than 10% w/w of the mixture of oligonucleotides, particularly less than 5% w/w of the mixture of oligonucleotides, and especially less than 2% w/w of the mixture of oligonucleotides.

The mass spectrometry employed in the present invention preferably comprises so-called soft ionisation techniques, such as Atmospheric Pressure Chemical Ionisation or especially electrospray ionisation.

In many preferred embodiments, the nature of the mobile phase in the desalting step is selected such that the charge state of the oligonucleotides produced by the mass spectrometer is collapsed into a single or a predominant single charge state. Preferred charge states are −3, −4, and −5.

In other embodiments, the nature of the mobile phase in the desalting step is selected such that the charge state of the oligonucleotides produced by the mass spectrometer is not collapsed into a single, or predominantly single charge state, and therefore remains in a multiple charge state. Examples of such conditions include the use of mildly acidic volatile compounds, such as halogenated C₁₋₄ alcohols, especially polyfluorinated alcohols such as hexafluoroisopropanol to form ammonium salts in the desalting step. Such conditions are particularly beneficial for the analysis of duplex oligonucleotides.

Quantitative analysis of the oligonucleotide mixture can be carried out by comparing the mass spectrum peak area or height for a given peak with the area or height from the peak of a known amount of sample. This may be done through extracted ion chromatograms of one or more charged state ions, with or without deconvolution, or the data may be smoothed and centroided, ie area or height data reduced to a vertical line. In certain embodiments, for impurities of similar molecular weight to the desired product, the response factor for the impurity and the product can be assumed to be the same. Accordingly, a calibration plot for either a known impurity or for the product can be employed for quantification. For impurities which have different response factors, a calibration plot for that impurity can be generated, or the quantity of impurity determined by addition of known amounts of the specific impurity and plotting the response versus quantity.

The method of the present invention is particularly suited to the quantitative analysis of synthetic oligonucleotides, and especially the rapid profiling of impurities in the analysis of multiple batches of synthetic oligonucleotides.

The present invention is illustrated, without limitation, by the following examples.

### EXAMPLE 1

### Preparation of Mobile Phase A (5 mM DMBAA, 5% ACN)

Glacial acetic acid (0.3 mL), dimethylbutylamine (0.7 mL), and acetonitrile (50 mL) were dissolved in water and brought to a volume of one litre.

### Preparation of Mobile Phase B (5mM DMBAA, 80% ACN)

Glacial acetic acid (0.3 mL), dimethylbutylamine (0.7 mL), and acetonitrile (800 mL) were dissolved in water and brought to a volume of one litre.

### Instrument:

Waters ZQ2000 mass spectrometer plus Waters Alliance 2795 RPLC plus 996 PDA, fitted with Waters XBridge™ 2.5 micron column, dimensions 4.6 mm×50 mm.

### Parameters:

Polarity: negative ESI

Capillary: 3 kV

Cone: 25V

Extractor: 3V

RF Lens: 0.5V

Source temp: 100° C.

Desolvation temp: 400° C.

Cone Gas Flow. 20 L/hr

Desolvation Gas Flow 790L/hr

LM 1 resolution: 15

HM 1 Resolution: 15

Ion Energy 1: 0

Multiplier: 650V

Mass Range: 1500-1900

Wavelength: 250-270 nm

Gradient Table:

| Time | % A | % B | Flow | Curve |
|---|---|---|---|---|
| 0.0 | 100 | 0 | 1.0 | 6 |
| 1.9 | 100 | 0 | 1.0 | 6 |
| 2.0 | 30 | 70 | 1.0 | 6 |
| 2.9 | 30 | 70 | 1.0 | 6 |
| 3.0 | 100 | 0 | 0.5 | 6 |
| 4.0 | 100 | 0 | 0.5 | 6 |
| 4.1 | 100 | 0 | 1.0 | 6 |
| 5.0 | 100 | 0 | 1.0 | 6 |

### Oligonucleotide Samples

A 1 mg/ml solution of a fully deprotected 22-mer phosphorothioate oligonucleotide (prepared by solid phase phosphoramidite chemistry and having a molecular weight of 7043) in Mobile phase A was loaded onto the RPLC column and eluted using the stepped conditions set out in the gradient table above. Elution of the oligonucleotide was detected by uv, and the eluent was analysed by the mass spectrometer.

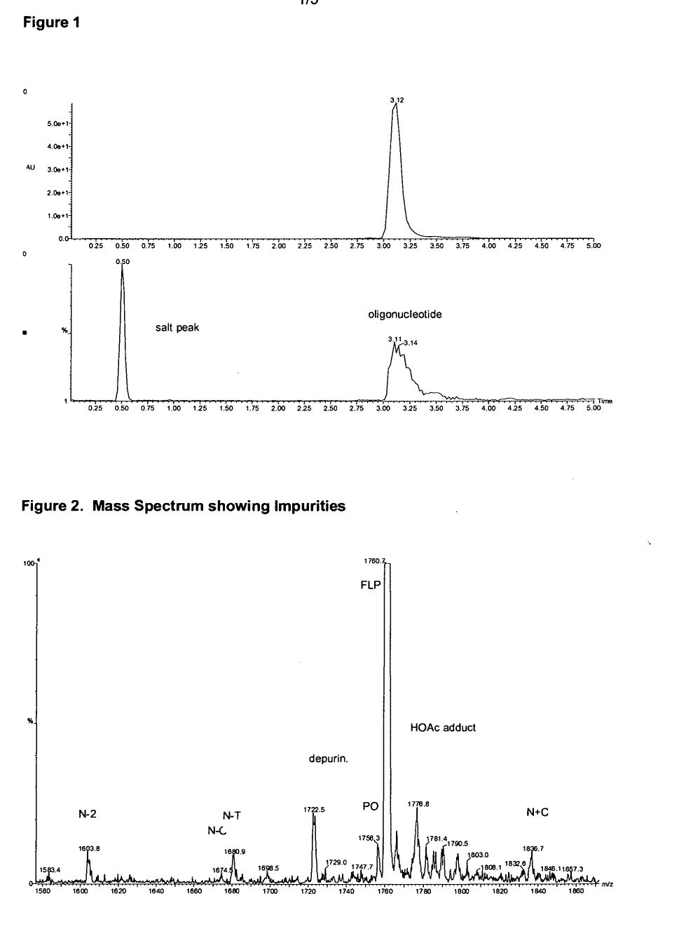

The efficient desalting achieved is shown by FIG. 1, where the top chromatogram shows the uv spectrometer trace, and the bottom chromatogram shows the mass spectrometer trace. The salt peak is clearly shown eluting at 0.5 minutes on the mass spectrometer trace.

Analysis of the oligonucleotide peak over the period from 3 to 3.75 minutes using the mass spectrometer software produced the Mass Spectrum shown in FIG. 2. The data indicated that the mixture of oligonucleotides was predominantly in the −4 charge state.

The analysis of the oligonucleotide sample was repeated two further times.

The analytical method was repeated, again in triplicate, for samples of the oligonucleotide spiked with known amounts of N-2 impurity missing the two 5- terminal nucleotides (T and C) compared with the full length oligonucleotide. Samples containing 1%, 3% and 5% w/w of N-2 impurity were prepared. Plotting the average peak areas for each sample expressed as the percentage of measured spike produced the graph.

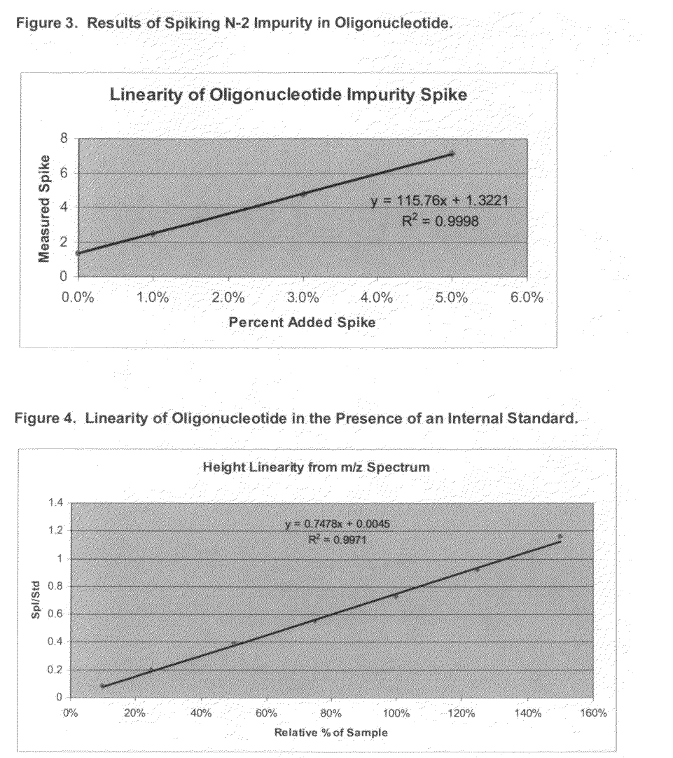

FIG. 3 shows that the amount of N-2 impurity present in the sample was 1.3%

### EXAMPLE 2

### Preparation of Mobile Phase A (5 mM DMBAA, 3% ACN)

Glacial acetic acid (0.3 mL), dimethylbutylamine (0.7 mL), and acetonitrile (30 mL) were dissolved in water and brought to a volume of one litre.

### Preparation of Mobile Phase B (5 mM DMBAA, 40% ACN)

Glacial acetic acid (0.3 mL), dimethylbutylamine (0.7 mL), and acetonitrile (400 mL) were dissolved in water and brought to a volume of one litre.

The instrument and column employed were as described in Example 1.

### Parameters:

Polarity: Negative ESI

Capillary: 3 kV

Cone: 25V

Extractor: 3V

RF Lens: 0.5V

Source temp: 130° C.

Desolvation temp: 400° C.

Cone Gas Flow. 60 L/hr

Desolvation Gas Flow 600 L/hr

LM 1 resolution: 15

HM 1 Resolution: 15

Ion Energy 1: 0.5

Multiplier: 650V

Mass Range: 1350-2000

Wavelength: 250-270 nm

Gradient Table:

| Time | % A | % B | Flow | Curve |
|---|---|---|---|---|
| 0.0 | 100 | 0 | 0.7 | 6 |
| 0.2 | 100 | 0 | 0.7 | 6 |
| 0.3 | 0 | 100 | 0.7 | 6 |
| 1.9 | 0 | 100 | 0.7 | 6 |
| 2.0 | 0 | 100 | 0.1 | 6 |
| 4.0 | 0 | 100 | 0.1 | 6 |
| 4.1 | 100 | 0 | 0.1 | 6 |
| 4.8 | 100 | 0 | 0.1 | 6 |
| 4.9 | 100 | 0 | 0.7 | 6 |
| 5.0 | 100 | 0 | 0.7 | 6 |

### Oligonucleotide Samples

A series of samples were prepared containing as an internal standard of 1 mg/mL aqueous solution of a 25-mer phosphorothioate oligonucleotide (molecular weight of 7776). The samples contained the oligonucleotide of interest, a 22-mer phosphorothioate oligonucleotide (molecular weight of 7048) in proportions varying from 150% to 10% relative to the internal standard. The samples were analyzed in triplicate using the RPLC column and the stepped conditions set out in the gradient table above.

The signal intensities for the −4 charge state for the sample and the −4 and −5 charge states for the internal standard were used in the evaluation. At each concentration level the intensities of each mass in the triplicate runs were averaged and the average intensity ratios of sample to standard were plotted with respect to the sample concentration, FIG. 4.

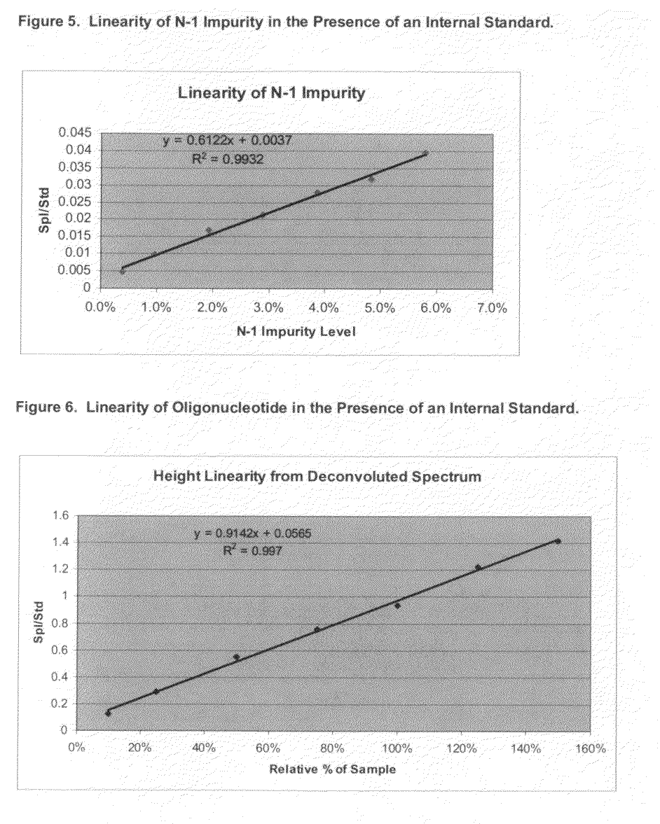

A similar treatment may be used for the impurities (such as PO and N-1 species) that normally are present in an oligonucleotide sample. Linearity is demonstrated at low levels, FIG. 5, and these impurities can also act as surrogates for the main peak when quantifying at low levels.

### EXAMPLE 3

### Preparation of Mobile Phase A (1% HFIP, 0.2% TEA, 3% ACN)

Hexafluoroisopropanol (10.0 mL), triethylamine (2.0 mL), and acetonitrile (30 mL) were dissolved in water and brought to a volume of one litre.

### Preparation of Mobile Phase B (1% HFIP, 0.2% TEA, 12% ACN)

Hexafluoroisopropanol (10.0 mL), triethylamine (2.0 mL), and acetonitrile (120 mL) were dissolved in water and brought to a volume of one litre.

The instrument and column employed were as described in Example 1

### Parameters:

Polarity: negative ESI

Capillary: 3 kV

Cone: 10V

Extractor: 3V

RF Lens: 0.5V

Source temp: 130° C.

Desolvation temp: 400° C.

Cone Gas Flow. 60 L/hr

Desolvation Gas Flow: 600 L/hr

LM 1 resolution: 15

HM 1 Resolution: 15

Ion Energy 1: 0.5

Multiplier: 650V

Mass Range: 602-2000

Wavelength: 250-270 nm

Gradient Table:

| Time | % A | % B | Flow | Curve |
|---|---|---|---|---|
| 0.0 | 100 | 0 | 0.7 | 6 |
| 0.3 | 100 | 0 | 0.7 | 6 |
| 0.4 | 0 | 100 | 0.7 | 6 |
| 2.2 | 0 | 100 | 0.7 | 6 |
| 2.3 | 0 | 100 | 0.2 | 6 |
| 4.8 | 0 | 100 | 0.2 | 6 |
| 4.9 | 100 | 0 | 0.7 | 6 |
| 5.0 | 100 | 0 | 0.7 | 6 |

### Oligonucleotide Samples

A series of samples were prepared containing as an internal standard of 1 mg/mL aqueous solution of a 25-mer phosphorothioate oligonucleotide (molecular weight of 7776). The samples contained the oligonucleotide of interest, a 22-mer phosphorothioate oligonucleotide (molecular weight of 7048) in proportions varying from 150% to 10% relative to the internal standard. The samples were analyzed in triplicate using the RPLC column and the stepped conditions set out in the gradient table above.

The m/z spectra were deconvoluted using the MaxEnt function of the software. At each concentration level the intensities of each deconvoluted mass in the triplicate runs were averaged and the average intensity ratios of sample to standard were plotted with respect to the sample concentration, FIG. 6.

### EXAMPLE 4

### Preparation of Mobile Phase A (5 mM DMBAF, 3% ACN)

Formic acid 88%(0.22 mL), dimethylbutylamine (0.7 mL), and acetonitrile (30 mL) were dissolved in water and brought to a volume of one litre.

### Preparation of Mobile Phase B (5 mM DMBAF, 40% ACN)

Formic acid 88% (0.22 mL), dimethylbutylamine (0.7 mL), and acetonitrile (400 mL) were dissolved in water and brought to a volume of one litre.

The instrument and column employed were as described in Example 1

### Parameters:

Polarity: negative ESI

Capillary: 3 kV

Cone: 10V

Extractor: 3V

RF Lens: 0.5V

Source temp: 130° C.

Desolvation temp: 400° C.

Cone Gas Flow. 60 L/hr

Desolvation Gas Flow: 600 L/hr

LM 1 resolution: 15

HM 1 Resolution: 15

Ion Energy 1: 0.1

Multiplier: 650V

Mass Range: 602-2000

Wavelength: 250-270 nm

Gradient Table:

| Time | % A | % B | Flow | Curve |
|---|---|---|---|---|
| 0.0 | 100 | 0 | 0.7 | 6 |
| 0.2 | 100 | 0 | 0.7 | 6 |
| 0.3 | 0 | 100 | 0.7 | 6 |
| 1.9 | 0 | 100 | 0.7 | 6 |
| 2.0 | 0 | 100 | 0.1 | 6 |
| 4.0 | 0 | 100 | 0.1 | 6 |
| 4.1 | 100 | 0 | 0.1 | 6 |
| 4.8 | 100 | 0 | 0.1 | 6 |
| 4.9 | 100 | 0 | 0.7 | 6 |
| 5.0 | 100 | 0 | 0.7 | 6 |

### Oligonucleotide Samples

A 1 mg/ml aqueous solution of a 58-mer phosphodiester oligonucleotide with a terminal phosphate group (molecular weight of 17950) was loaded onto the RPLC column and eluted using the stepped conditions set out in the gradient table above. Elution of the oligonucleotide was detected by uv, and the eluent was analysed by the mass spectrometer.

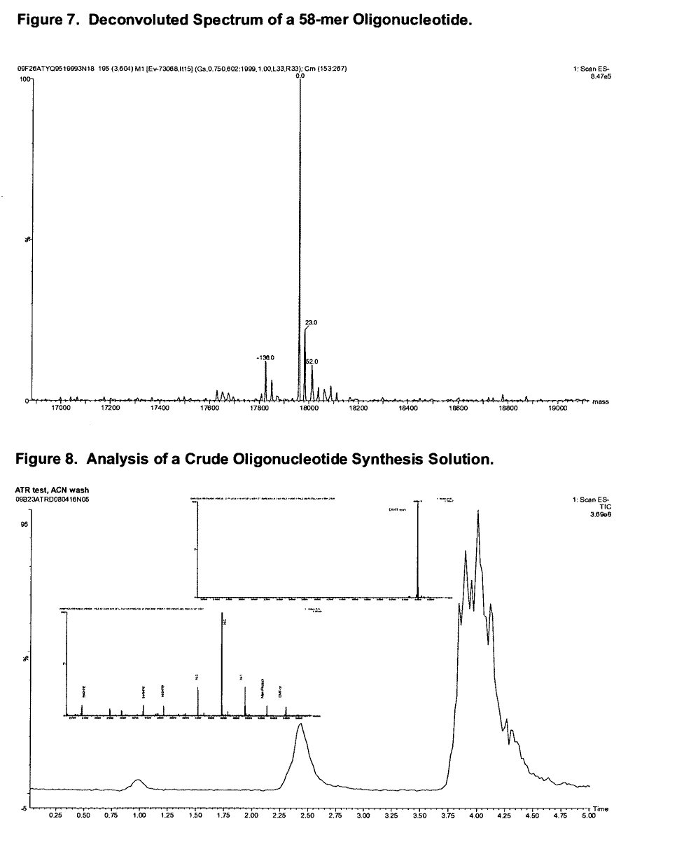

The m/z spectra was deconvoluted using the MaxEnt function of the software. The molecular weight of the oligonucleotide, as well as its impurities and adducts are shown in FIG. 7.

### EXAMPLE 5

The instrument and column employed were as described in Example 1.

Mobile phases A and B were prepared as described in Example 3 Mobile phase C consisted of acetonitrile.

### Parameters:

Polarity: negative ESI

Capillary: 3 kV

Cone: 10V

Extractor: 3V

RF Lens: 0.5V

Source temp: 130° C.

Desolvation temp: 400° C.

Cone Gas Flow. 60 L/hr

Desolvation Gas Flow. 600 L/hr

LM 1 resolution: 15

HM 1 Resolution: 15

Ion Energy 1: 0.1

Multiplier: 650V

Mass Range: 602-2000

Wavelength: 250-270 nm

Gradient Table:

| Time | % A | % B | % C | Flow | Curve |
|---|---|---|---|---|---|
| 0.0 | 100 | 0 | 0 | 0.7 | 6 |
| 0.5 | 100 | 0 | 0 | 0.7 | 6 |
| 0.6 | 0 | 100 | 0 | 0.7 | 6 |
| 2.0 | 0 | 100 | 0 | 0.7 | 6 |
| 2.1 | 0 | 50 | 50 | 0.7 | 6 |
| 4.8 | 0 | 50 | 50 | 0.7 | 6 |
| 4.9 | 100 | 0 | 0 | 0.7 | 6 |
| 5.0 | 100 | 0 | 0 | 0.7 | 6 |

### Oligonucleotide Samples

A crude synthesis solution of a 16-mer phosphorothioate oligonucleotide containing some locked nucleic acids (LNA) was loaded onto the RPLC column and eluted using the stepped conditions set out in the gradient table above. Elution of the oligonucleotide was detected by uv, and the eluent was analysed by the mass spectrometer.

The dimethoxytrityl protecting group (DMT) is normally attached to the full length oligonucleotide at the completion of synthesis. This hydrophobic group required an extra step of 50% acetonitrile for elution. FIG. 8 clearly shows the elution of typical shortmer impurities (DMT-off) followed by the main oligonucleotide (DMT-on).

### EXAMPLE 6

The instrument and column employed were, as described in Example 1. The mobile phases were prepared as in Example 3

### Parameters:

Polarity: negative ESI

Capillary: 3 kV

Cone: 10V

Extractor: 3V

RF Lens: 0.5V

Source temp: 130° C.

Desolvation temp: 400° C.

Cone Gas Flow. 60 L/hr

Desolvation Gas Flow 600 L/hr

LM 1 resolution: 15

HM 1 Resolution: 15

Ion Energy 1: 0.0

Multiplier: 650V

Mass Range: 602-1400

Wavelength: 250-270 nm

Gradient Table:

| Time | % A | % B | Flow | Curve |
|---|---|---|---|---|
| 0.0 | 100 | 0 | 1.0 | 6 |
| 1.0 | 100 | 0 | 1.0 | 6 |
| 1.1 | 0 | 100 | 1.0 | 6 |
| 2.0 | 0 | 100 | 1.0 | 6 |
| 2.1 | 100 | 0 | 0.5 | 6 |
| 3.0 | 100 | 0 | 0.5 | 6 |
| 3.1 | 100 | 0 | 1.0 | 6 |
| 5.0 | 100 | 0 | 1.0 | 6 |

### Oligonucleotide Samples

A 21-mer phosphodiester RNA oligonucleotide containing a both standard and 2′-OMethyl nucleotides was mixed with a 23-mer complementary RNA strand to induce duplex formation. The sample was loaded onto the RPLC column and eluted using the stepped conditions set out in the gradient table above. Elution of the oligonucleotide was detected by uv, and the eluent was analysed by the mass spectrometer.

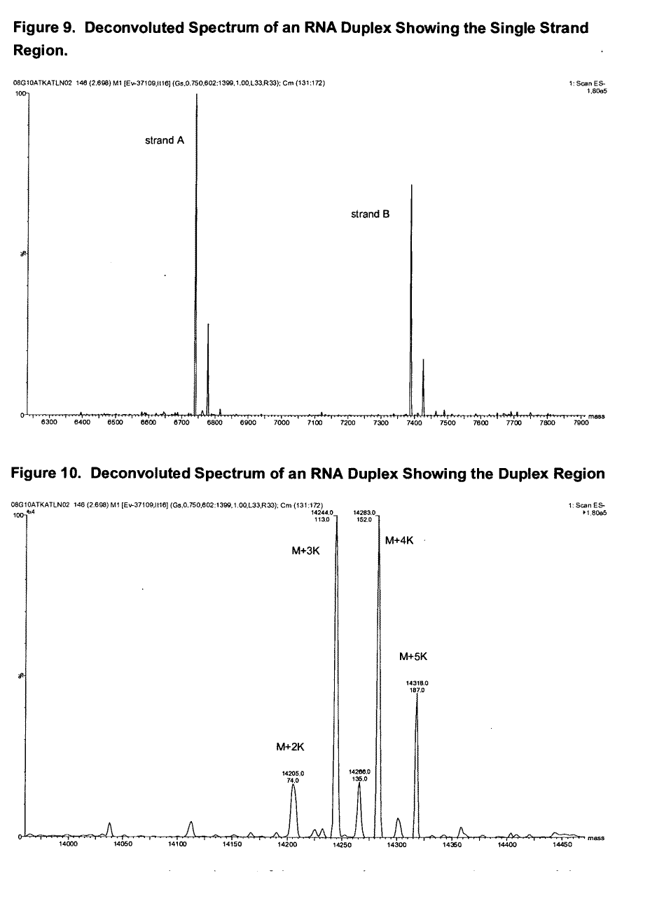

In FIGS. 9 and 10 respectively, both single strands are observed and the duplex is observed as a cluster of potassium adducts.

Examples 2 to 6 demonstrate how various species can be detected for quantitative analysis following the method of the present invention, for example using the quantification approach described in Example 1.

1. A method comprising:
a) desalting a mixture of oligonucleotides under conditions such that there is substantially no separation of the mixture of oligonucleotides;
b) introducing the desalted mixture of oligonucleotides into a mass spectrometer; and
c) quantifying one or more oligonucleotides comprised in the mixture by mass spectrometry.

2. A method according to claim 1, wherein at least 95%, and preferably at least 99% of the oligonucleotides coelute.

3. A method comprising:
a) on-line desalting a mixture of oligonucleotides;
b) introducing the desalted mixture of oligonucleotides into a mass spectrometer; and
c) quantifying one or more oligonucleotides comprised in the mixture by mass spectrometry.

4. A method according to claim 3, wherein the on-line desalting is achieved by chromatography under conditions such that there is substantially no separation of the mixture of oligonucleotides.

5. A method according to claim 4, wherein at least 95%, and preferably at least 99% of the oligonucleotides coelute.

6. A method according to any preceding claim wherein the mixture of oligonucleotides is desalted to the extent that non-volatile salt adducts comprise less than 20% w/w of the mixture of oligonucleotides, and preferably less than 5% w/w of the mixture of oligonucleotides

7. A method according to any preceding claim, wherein the mixture of oligonucleotides is desalted to the extent that sodium salt adducts comprise less than 2% w/w of the mixture of oligonucleotides.

8. A method according to any preceding claim, where the mixture of oligonucleotides is desalted by reverse phase liquid chromatography.

9. A method according to claim 8, wherein the reverse phase liquid chromatography comprises elution of the mixture of oligonucleotides by a mobile phase comprising an ammonium salt.

10. A method according to claim 9, wherein the ammonium salt is a C₁₋₄ alkyl tertiary ammonium salt, preferably a dimethylbutyl ammonium salt.

11. A method according to claim 10, wherein the ammonium salt is a carbonate, formate, acetate, trifluoroacetate or propionate salt.

12. A method according to any preceding claim, wherein the charge state of the mixture of oligonucleotides in the mass spectrometer is collapsed into a single or a predominant single charge state, and preferably −3, −4, and −5.

13. A method for preparing an oligonucleotide which comprises the steps of
a) synthesising an oligonucleotide, and
b) analysing the composition of said oligonucleotide;
wherein the oligonucleotide is analysed by a method as claimed in any one preceding claim.

14. A method according to claim 13, wherein the oligonucleotide is synthesised by solid phase phosphoramidite chemistry.

15. A method according to either of claims 13 and 14, wherein the oligonucleotide is a drug product.
